(19) United States
(12) Patent Application Publication
(10) Pub. No.: US 2024/0245592 A1
(43) Pub. Date: Jul. 25, 2024

(54) Emulsion or Oil-Dispersion Cosmetic Composition Containing High Content of Powder

(71) Applicant: LG Household & Health Care Ltd., Seoul (KR)

(72) Inventors: Il Shin Maeng, Seoul (KR); Jae Sik Han, Seoul (KR)

(73) Assignees: LG Household & Health Care Ltd., Seoul (KR); LG Household & Health Care Ltd., Seoul (KR)

(21) Appl. No.: 18/290,377

(22) PCT Filed: May 9, 2022
(86) PCT No.: PCT/KR2022/006547

§ 371 Date: Nov. 13, 2023

(30) Foreign Application Priority Data
May 18, 2021 (KR) 10-2021-0064228
Dec. 3, 2021 (KR) 10-2021-0171581

(51) Int. Cl. A61K 8/37; A61K 8/02; A61K 8/29; A61K 8/81; A61K 8/92; A61Q 19/00

(52) U.S. Cl. CPC A61K 8/37; A61K 8/0216; A61K 8/29; A61K 8/8111; A61K 8/92; A61Q 19/00; A61K 2800/10

(57) ABSTRACT

The present invention relates to an emulsion or oil-dispersion cosmetic composition comprising wax, a powder, and a fatty acid ester surfactant, wherein the powder is included in 20 wt % or more relative to the total weight of the composition, and can provide a composition which contains a high content of powder, has excellent dispersibility by preventing the agglomeration of the powder due to wax, has improved filling properties of the powder, and has excellent fluidity at a high temperature.

### TECHNICAL FIELD

The present invention relates to an emulsion or oil dispersion cosmetic composition comprising a high content of powder, and specifically, to a composition comprising a wax, a powder and a fatty acid ester surfactant.

### BACKGROUND ART

In order to realize smooth spreading and a powdery finish, a high content of volatile oil and powder can be used. However, oil dispersion balm formulations such as sticks have a limitation in use of volatile oils and a difficulty in realizing a feeling of use due to the limitation in formulation according to the sealing power of a container despite their advantage such as high portability and high convenience of use.

In particular, since solid balm-type cosmetics including a wax are filled at high temperature above the melting point for a long period of time, the inclusion of a high volatile oil content may affect the feeling of use and formulation stability of products before and after filling and according to a filling time.

Conventionally, it was intended to develop cosmetics in the form of an emulsion and oil dispersion balm including a high content of powder instead of volatile oil so as to overcome the limitation in formulation stability and container restrictions while realizing smooth spreading and a powdery finish. However, when a large amount of spherical powder was applied, there was a problem in that, as the powder content increased, powder dispersibility and high-temperature fluidity were degraded, and thus productivity was lowered in the filling process. When a bentonite-based raw material for improving dispersibility and a surfactant were used to increase fluidity, there was a problem in that the viscosity of a formulation was increased to degrade fluidity, which further adversely affected productivity.

### DISCLOSURE

### Technical Problem

The present inventors have found that, in an emulsion and oil dispersion formulation including a wax, there is a strong tendency for powder particles to agglomerate with each other due to the structure of the wax, but the powder agglomeration can be prevented by applying a fatty acid ester surfactant.

Accordingly, the present invention is directed to providing an emulsion or oil dispersion cosmetic composition, which has improved powder dispersibility and is able to exhibit excellent fluidity at high temperature while including a high content of powder.

### Technical Solution

One aspect of the present invention provides an emulsion or oil dispersion cosmetic composition comprising a wax, a powder and a fatty acid ester surfactant, wherein the powder is included in an amount of 20 wt % or more relative to the total weight of the composition.

### Advantageous Effects

The present invention can provide a composition which has excellent dispersibility by preventing powder agglomeration caused by a wax, improved powder fillability by having a high powder content, and excellent fluidity at high temperature while including a high content of powder.

In addition, the composition according to the present invention can have a skin improvement effect by providing excellent spreadability and a soft feeling of use.

### DESCRIPTION OF DRAWINGS

FIG. 1 is a schematic diagram of dispersion of a surfactant and a powder in a wax according to the present invention.

FIG. 2 is a set of images showing high-temperature fluidity according to Experimental Example 1, FIG. 2A represents Example 1, FIG. 2B represents Example 2, FIG. 2C represents Comparative Example 1, and FIG. 2D represents Example 3.

FIG. 3 is a set of images showing high-temperature fluidity according to Experimental Example 4, and FIG. 3A represents Example 4, FIG. 3B represents Example 5, and FIG. 3C represents Comparative Example 2.

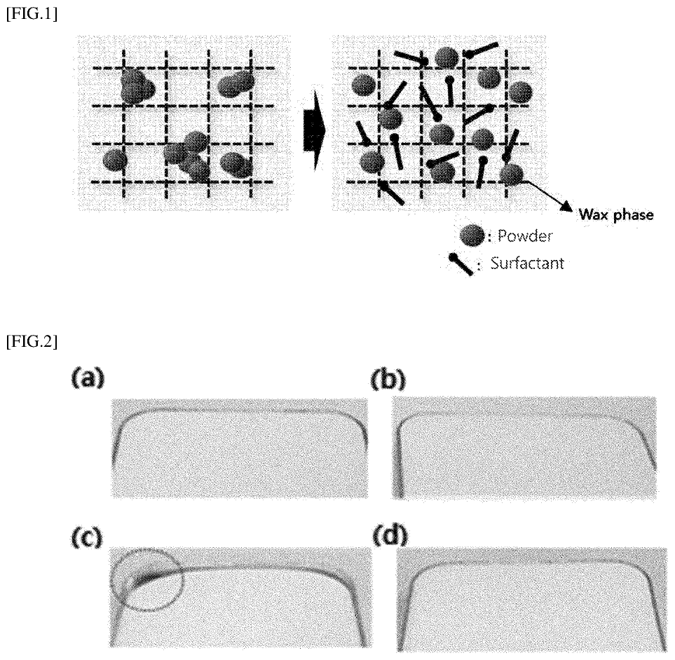

### MODES OF THE INVENTION

Hereinafter, the present invention will be described in detail.

The present invention relates to an emulsion or oil dispersion cosmetic composition comprising a wax, a powder and a fatty acid ester surfactant, wherein the powder is included in an amount of 20 wt % or more relative to the total weight of the composition.

The inventors of the present invention have found that, when a fatty acid ester surfactant with an ester condensation structure having one or more hydrophilic groups including a hydroxyl group and one or more lipophilic groups including a fatty acid is used, powder agglomeration is prevented to improve dispersibility, and fillability at high temperature is improved, and thus a high content of powder can be included in a formulation.

Specifically, powder particles have a strong tendency to agglomerate with each other due to the structure of a wax, but the surfactant according to the present invention not only exhibits better compatibility with a wax but also has a larger volume than other surfactants by including a fatty acid, and thus it may physically disperse a powder and maintain that state.

In the present invention, “high temperature” refers to a temperature of 40° C. or more, and more specifically, a temperature of 45° C. or more, 50° C. or more or 55° C. or more.

In the present invention, the surfactant is a fatty acid ester surfactant and refers to a product of an ester condensation reaction between a hydroxyl group and a carboxyl group of a fatty acid.

The surfactant of the present invention may be specifically formed by an ester condensation reaction between a polyalcohol and a fatty acid or an ester condensation reaction of only a fatty acid compound including both a hydroxyl group and a carboxyl group.

Since the surfactant of the present invention has a large volume by including a fatty acid, it may physically disperse a powder. Examples of the fatty acid include lauric acid, myristic acid, palmitic acid, stearic acid, nonadecylic acid, hydroxystearic acid and hydroxymyristic acid.

In an embodiment of the present invention, the surfactant may be one or more selected from the group consisting of polyhydroxy fatty acids, polyethylene glycol fatty acid esters, glycerin fatty acid esters and polyglycerol fatty acid esters.

The polyhydroxy fatty acid is a polymer of a fatty acid including a hydroxyl group and may be formed by an intramolecular ester condensation polymerization reaction due to the hydroxyl group in the fatty acid. For example, polyhydroxystearic acid is a polymer of hydroxystearic acid and refers to a polymer formed by an ester condensation polymerization reaction of hydroxystearic acid.

The polyethylene glycol fatty acid ester may be formed by an ester condensation polymerization reaction between a hydroxyl group of polyethylene glycol and a fatty acid. For example, PEG-30 dipolyhydroxystearate may refer to a diester of 30 repeating intramolecular ethylene oxide units and hydroxystearate.

The glycerin fatty acid ester may be formed by an ester condensation polymerization reaction between glycerin and a fatty acid, and the polyglycerol fatty acid ester may be formed by an ester condensation polymerization reaction between polyglycerol and a fatty acid.

In the present invention, as the surfactant, one or more selected from the group consisting of polyhydroxystearic acid, PEG-30 dipolyhydroxystearate, PEG-20 dipolyhydroxymyristate, PEG-40 dipolyhydroxypalmitate, PEG-4 trimethylolpropane distearate, PEG-4 glyceryl distearate, polyglyceryl-2 diisostearate, polyglyceryl-diisostearate, polyglyceryl-3 distearate, polyglyceryl-3 polyricinoleate, PEG-6 distearate, PEG-6 diisostearate, PEG-8 distearate, PEG-8 diisostearate, PEG-12 diisostearate, glyceryl stearate, PEG-23 stearate, PEG-40 stearate, PEG-100 stearate, PEG-150 stearate, sorbitan stearate, sorbitan sesquioleate and sorbitan olivate may be used, but the present invention is not limited thereto.

More preferably, the surfactant of the present invention may be one or more selected from the group consisting of polyhydroxystearic acid, PEG-30 dipolyhydroxystearate and a mixture thereof.

In an embodiment of the present invention, the surfactant may have a molecular weight of 2,000 to 100,000 g/mol. When the molecular weight falls within the above range, the surfactant can easily physically disperse the powder.

In an embodiment of the present invention, the surfactant may be included in an amount of 0.01 to 3 wt % relative to the total weight of the composition. Preferably, the surfactant may be included in an amount of 0.01 to 2 wt %, 0.01 to 1 wt %, 0.1 to 1 wt % or 0.1 to 0.8 wt %. When the content of the surfactant falls within the above range, the powder can be sufficiently dispersed, and high-temperature fluidity can be improved.

In the present invention, the wax may be one or more selected from the group consisting of ozokerite, beeswax, cocoa butter, candelilla wax, carnauba wax, synthetic wax, microcrystalline wax, ceresin wax, polyethylene wax and beeswax. Most preferably, the wax may be ceresin wax or synthetic wax. However, any wax may be used without limitation as long as it is used in preparation of a balm formulation or stick formulation in the art.

In an embodiment of the present invention, the wax may be included in an amount of 2 to 10 wt % relative to the total weight of the composition. Preferably, the wax may be included in an amount of 2 to 8 wt % or 4 to 8 wt %. When the content of the wax is less than 2 wt %, viscosity is insufficient, and thus it may be difficult to prepare a desired cosmetic formulation, and when the content exceeds 10 wt %, formulation stability or feeling of use may be degraded.

In the present invention, the powder may be one or more selected from titanium dioxide, zinc oxide, talc, mica, iron oxide, nylon, polymethylsilsesquioxane, synthetic fluorphlogopite, a methyl methacrylate crosspolymer, silica and a mixture thereof. Preferably, the powder may be titanium dioxide, polymethylsilsesquioxane or a mixture thereof.

The powder may be included in an amount of 20 wt % or more relative to the total weight of the composition. Preferably, the powder may be included in an amount of 30 wt % or more, 40 wt % or more, 43 wt % or more, 45 wt % or more or 50 wt % or more, and more preferably, 30 to 80 wt %, 40 to 80 wt %, 45 to 75 wt %, 45 to 70 wt % or 50 to 70 wt %. The composition of the present invention may have high fluidity and fillability while including a high content of the powder within the above content range. The powder is not particularly limited to the content or type of the powder as long as it provides a powdery feeling of use.

In the present invention, the powder may have a size of 0.1 to 100 μm, preferably 0.1 to 10 μm, and more preferably 0.1 to 1 μm or 3 to 10 μm. For example, the powder may have a size of about 250 nm or 4 to 9 μm.

The composition of the present invention may further include an oil phase. Also, a weight ratio of the oil phase and the powder may be 1:1 to 1:4. Preferably, the weight ratio may be 1:1 to 1:3, and more preferably, 1:1.3 to 1:2.5. When the oil phase and the powder are included in the above weight ratio, smooth spreading and a powdery feeling of use may be provided without leaving a sticky feeling.

The oil phase may include one or more selected from the group consisting of a silicone oil, an ester-based oil, a hydrocarbon-based oil, a triglyceride-based oil and a mixture thereof.

The silicone oil may be one or more selected from the group consisting of dimethicone, cyclomethicone, polydimethylsiloxane, methylphenylpolysiloxane, methylcyclopolysiloxane, octamethylcyclotetrasiloxane, decamethylcyclopentasiloxane, dodecamethylcyclohexasiloxane, tetradecamethylhexasiloxane and octamethyltrisiloxane, but the present invention is not limited thereto.

The ester-based oil may be one or more selected from the group consisting of hexyl laurate, triethylhexanoin, cetyl ethylhexanoate, cetyl octanoate, cetyl isooctanoate, octyldodecyl myristate, pentaerythrityl tetraethylhexanoate, isopropyl palmitate, isopropyl myristate, caprylic/capric triglyceride, butylene glycol dicaprylate/dicaprate, tocopherol acetate and dicaprylic carbonate, but the present invention is not limited thereto.

The hydrocarbon-based oil may be one or more selected from the group consisting of paraffin, ceresin and microcrystalline wax, but the present invention is not limited thereto.

The triglyceride-based oil may be one or more selected from the group consisting of C8-12 acid triglyceride, C12-18 acid triglyceride, caprylic/capric triglyceride, caprylic/capric/lauric triglyceride, C10-40 isoalkyl acid triglyceride, C10-18 triglyceride, glyceryl triacetyl hydroxystearate, soybean glyceride, tribehenin, tricaprin, triethylhexanoin, triheptanoin, triisostearin, tripalmitin and tristearin, but the present invention is not limited thereto.

The composition of the present invention may have a hardness of 100 to 5,000 dyne/cm², preferably 200 to 4000 dyne/cm², and more preferably 1500 to 3200 dyne/cm² when in a solid state such as a balm formulation at room temperature, but the present invention is not limited thereto. The hardness may be measured, for example, at a speed of 2 cm/min and a depth of 2 mm using a pin having a diameter of 10 Φ in a dedicated adapter of a Fudoh rheometer. Preferably, the composition of the present invention may be in the form of a balm having a hardness of 100 to 5,000 dyne/cm².

The composition of the present invention may have a viscosity of 10 to 1000 centipoise (cps), preferably 10 to 200 cps, 15 to 200 cps or 10 to 100 cps, and most preferably 13 to 90 cps. The above viscosity range may be measured at high temperature, preferably a temperature of 60 to 100° C., and more preferably a temperature of 70 to 90° C., for example, at 80° C. When the high-temperature viscosity of the composition is less than 10 cps, formulation stability in the molten state is degraded due to excessive flowability, and when the high-temperature viscosity exceeds 1000 cps, fillability is poor due to excessive viscosity, and it is difficult to produce a desired shape. The viscosity may be measured using spindle #3 in a Brookfield rheometer at 30 rpm for 1 minute.

The composition of the present invention may further include a typically used emulsion stabilizer, UV protection agent, moisturizer, oil-phase base, preservative, antioxidant, alcohol, fragrance, pH control agent or natural extract, but the present invention is not limited thereto.

The composition according to the present invention may be substantially or completely free of a thickener. Particularly, a powder-based oil-phase thickener such as hectorite may not be substantially included. Also, the composition according to the present invention may be substantially or completely free of a volatile oil.

The composition according to the present invention may be in the form of a non-aqueous oil dispersion that is substantially or completely free of an aqueous phase.

In the present invention, “substantially free of” may refer to being included at less than 1 wt % or less than 0.1 wt % or not included at all.

The composition of the present invention may be prepared into a balm formulation, particularly, an oil dispersion balm formulation, and when the composition is prepared into the above formulation, the viscosity (fluidity) of the composition is excellent in a high-temperature preparation process, and thus excellent fillability may be exhibited.

The balm formulation refers to a form that is present in a solid state and melts when applied on the skin, but the present invention is not limited thereto. The balm formulation may be formulated as oil balm, cleansing balm, lip balm, sun balm, hair balm, multi balm or the like.

In addition, the composition according to the present invention may be prepared into a stick, cake, compact or gel formulation in addition to the balm formulation and may be used for any one purpose selected from the group consisting of a foundation, a concealer, a makeup base, a BB cream, a lotion, a cream and a wax.

Hereinafter, the present invention will be described in detail with reference to the following experimental examples. However, it should be understood that the following experimental examples are given for the purpose of illustration only and are not intended to limit the scope of the present invention. In addition, these experimental examples are only intended to aid in understanding the present invention, and the scope of the present invention is not limited to the following experimental examples.

### EXAMPLES

### Preparation Example 1—Preparation of Compositions of Examples 1 to 3 and Comparative Example 1

A wax ingredient, an oil phase ingredient and a surfactant were input into an oil phase tank, heated to 85° C. so that the wax completely melted, and then homogeneously mixed using a disper mixer. After homogeneous mixing was confirmed, a powder was slowly input and completely dispersed using a disper mixer, and defoaming was performed to prepare a composition. Compositions of Examples 1 to 3 and Comparative Example 1 were prepared with compositions described in the following Table 1.

TABLE 1

| | | Example | Example | Comparative | Example |
|---|---|---|---|---|---|
| Classification | Ingredient name | 1 | 2 | Example 1 | 3 |
| Wax | Ceresin | 3.5 | 3.5 | 3.5 | 3.5 |
| ingredient | Synthetic wax, | 2.5 | 2.5 | 2.5 | 2.5 |
| | ethylene/propylene copolymer | | | | |
| Oil phase | Butylene glycol | to 100 | to 100 | to 100 | to 100 |
| ingredient | dicaprylate/dicaprate | | | | |
| | Hexyl laurate | 5 | 5 | 5 | 5 |
| | Dimethicone | 5 | 5 | 5 | 5 |
| Surfactant | Polyhydroxystearic acid | 0.3 | 0.3 | - | 0.6 |
| Powder | Titanium dioxide | 25 | 25 | 25 | 25 |
| | Polymethylsilsesquioxane | 32 | 35 | 38 | 38 |
| Preservative | 1,2-Hexanediol | 0.5 | 0.5 | 0.5 | 0.5 |

*Hexyl laurate: KAK-HL (trade name, commercially available from Kokyu Alcohol Kogyo Co., Ltd.)
*Butylene glycol dicaprylate/dicaprate: Dermofeel ® BGC (trade name, commercially available from Dr. Straetmans)

As polyhydroxystearic acid, Salacos HS-6C commercially available from Nisshin Oillio was used. As titanium dioxide in the powder, LP-TR-10 commercially available from Miyoshi was used, and the average size thereof was 250 m. As polymethylsilsesquioxane, SESQ-101 commercially available from N&M Tech was used, and the average size thereof was 4 to 9 μm.

### Experimental Example 1—Evaluation of High-Temperature Fluidity

Each composition according to Preparation Example 1 was filled (15 g) at 80° in an x-1 metal plate, whether the content was piled up or sufficiently filled the edges was visually compared, and a smoother surface and a smaller empty space at the edge were judged to be better fluidity. Results thereof are shown in the following Table 2 and FIG. 2. FIGS. 2A to 2D represent Example 1, Example 2, Comparative Example 1 and Example 3, respectively.

TABLE 2

| | | | Comparative | |
|---|---|---|---|---|
| Classification | Example 1 | Example 2 | Example 1 | Example 3 |
| High- | ⊚ | ◯ | X | ⊚ |
| temperature | | | | |
| fluidity | | | | |

As shown in FIG. 2 and Table 2, high-temperature fluidity was degraded as the powder content increased, and high-temperature fluidity was improved as the surfactant content increased. Particularly, Comparative Example 1 not including a surfactant showed that the content was piled up and did not fill the edge, whereas Example 3 showed that, despite a high powder content, the formulation filled the edge of the metal plate without an empty space, and the surface was smooth, and thus high-temperature fluidity was significantly improved.

### Experimental Example 2—Sensory Evaluation

5-point sensory evaluation about spreadability, coverage, powderiness and preference was conducted with 20 consumers in their 20s to 40s. Results thereof are shown in Table 3.

TABLE 3

| | | Comparative | |
|---|---|---|---|
| Classification | Example 1 | Example 1 | Example 3 |
| Spreadability* | 4.8 | 2.2 | 4.8 |
| Coverage* | 4.5 | 4.4 | 4.6 |
| Powderiness* | 3.5 | 4.5 | 4.6 |
| Preference* | 4.2 | 2.3 | 4.6 |

The coverage score was high in all samples due to the high titanium dioxide content. Also, when Examples 1 and 3 were compared, as the powder content increased, spreadability was maintained, but the powderiness score increased. When Comparative Example 1 and Example 3 were compared, when the same powder content was used, as the surfactant content increased, high-temperature fluidity was improved, and the spreadability and powderiness scores also increased.

### Experimental Example 3—Measurement of Viscosity and Hardness

Hardness and viscosity according to Examples 1 to 3 and Comparative Example 1 were measured. Hardness was determined by filling the composition at 80° in an x-1 metal plate as in Experimental Example 1 and, 1 hour later, measuring penetration hardness at a speed of 2 cm/min and a depth of 2 mm using a No. 3 (D=10φ) spindle in a Fudoh rheometer. Viscosity was measured using spindle #3 in a Brookfield rheometer at 80° C. and 30 rpm for 1 minute. Results thereof are shown in Table 4.

TABLE 4

| | | | Comparative | |
|---|---|---|---|---|
| Classification | Example 1 | Example 2 | Example 1 | Example 3 |
| Hardness | 1730 | 2100 | 2640 | 2450 |
| Viscosity | 21 | 62 | 126 | 15 |

As shown in Table 4, Examples 1 to 3 had excellent fillability by having lower viscosity values than Comparative Example 1 and exhibited appropriate levels of hardness.

### Preparation Example 2—Preparation of Compositions of Examples 4 and 5 and Comparative Example 2

Compositions of Examples 4 and 5 and Comparative Example 2 were prepared with compositions described in the following Table 5 in the same manner as in Preparation Example 1, except that PEG-30 dipolyhydroxystearate with a similar structure to polyhydroxystearic acid and PEG-10 dimethicone, which is a commonly used surfactant, were used in addition to polyhydroxystearic acid.

TABLE 5

| | | Example | Example | Comparative |
|---|---|---|---|---|
| Classification | Ingredient name | 4 | 5 | Example 2 |
| Wax | Ceresin | 3.5 | 3.5 | 3.5 |
| ingredient | Synthetic wax, | 2.5 | 2.5 | 2.5 |
| | ethylene/propylene copolymer | | | |
| Oil phase | Butylene glycol dicaprylate/dicaprate | to 100 | to 100 | to 100 |
| ingredient | Hexyl laurate | 5 | 5 | 5 |
| | Dimethicone | 5 | 5 | 5 |
| Surfactant | Polyhydroxystearic acid | 0.6 | — | — |
| | PEG-30 dipolyhydroxystearate | — | 0.6 | — |
| | PEG-10 dimethicone | — | — | 0.6 |
| Pigment | Titanium dioxide | 25 | 25 | 25 |
| | Polymethylsilsesquioxane | 38 | 38 | 38 |
| Preservative | 1,2-Hexanediol | 0.5 | 0.5 | 0.5 |

Salacos HS-6C commercially available from Nisshin Oillio was used as polyhydroxystearic acid, CITHROL DPHS-SO-(MV) (ARLACEL 135) commercially available from Croda was used as PEG-30 dipolyhydroxystearate, and KF-6017 commercially available from SHIN-ETSU was used as PEG-10 dimethicone. As titanium dioxide in the powder, LP-TR-10 commercially available from Miyoshi was used, and the average size thereof was 250 μm. As polymethylsilsesquioxane, SESQ-101 commercially available from N&M Tech was used, and the average size thereof was 4 to 9 km.

### Experimental Example 4—Evaluation of High-Temperature Fluidity

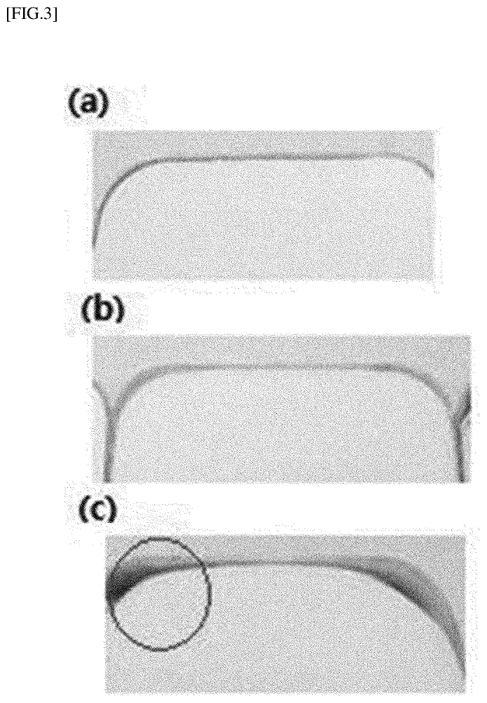

High-temperature fluidity was evaluated in the same manner as in Experimental Example 1. Results thereof are shown in the following Table 6 and FIG. 3. FIGS. 3A to 3C represent Example 4, Example 5 and Comparative Example 2, respectively.

TABLE 6

| | | | Comparative |
|---|---|---|---|
| Classification | Example 4 | Example 5 | Example 2 |
| High-temperature fluidity | ⊚ | ◯ | X |

As shown in FIG. 3 and Table 6, Example 5 using PEG-30 dipolyhydroxystearate as a surfactant exhibited improved high-temperature fluidity which was slightly degraded compared to Example 4, and there was no problem in the filling state. However, Comparative Example 2 using PEG-10 dimethicone as a surfactant exhibited degraded high-temperature fluidity, and the filling state was not uniform.

### Experimental Example 5—Measurement of Viscosity and Hardness

Hardness and viscosity according to Examples 4 and 5 and Comparative Example 2 were measured in the same manner as in Experimental Example 3, and results thereof are shown in Table 7.

TABLE 7

| | | | Comparative |
|---|---|---|---|
| Classification | Example 4 | Example 5 | Example 2 |
| Hardness (dyne/cm²) | 2450 | 3180 | 3090 |
| Viscosity | 15 | 68 | 98 |

As shown in Table 7, Examples 4 and 5 had excellent fillability by having lower viscosity values than Comparative Example 2 and exhibited appropriate levels of hardness.

1. An emulsion or oil dispersion cosmetic composition comprising a wax, a powder and a fatty acid ester surfactant,
wherein the powder is included in an amount of 20 wt % or more relative to the total weight of the composition.

2. The cosmetic composition of claim 1, wherein the surfactant is one or more selected from the group consisting of polyhydroxy fatty acids, polyethylene glycol fatty acid esters, glycerin fatty acid esters and polyglycerol fatty acid esters.

3. The cosmetic composition of claim 1, wherein the surfactant is one or more selected from the group consisting of polyhydroxystearic acid, PEG-30 dipolyhydroxystearate and a mixture thereof.

4. The cosmetic composition of claim 1, wherein the surfactant has a molecular weight of 2,000 to 100,000 g/mol.

5. The cosmetic composition of claim 1, wherein the wax is one or more selected from the group consisting of ozokerite, beeswax, cocoa butter, candelilla wax, carnauba wax, synthetic wax, microcrystalline wax, ceresin wax, polyethylene wax and beeswax.

6. The cosmetic composition of claim 1, wherein the powder is included in an amount of 30 to 80 wt % relative to the total weight of the composition.

7. The cosmetic composition of claim 1, wherein the powder has a size of 0.1 to 100 μm.

8. The cosmetic composition of claim 1, further comprising an oil phase,
wherein a weight ratio of the oil phase and the powder is 1:1 to 1:4.

9. The cosmetic composition of claim 1, wherein the composition is in a form of a balm having a hardness of 100 to 5,000 dyne/cm².

10. The cosmetic composition of claim 1, wherein the composition has a viscosity of 10 to 1000 cps at 60 to 100° C.

11. The cosmetic composition of claim 1, wherein the composition is in a form of a non-aqueous oil dispersion.
